(19) United States
(12) Patent Application Publication
(10) Pub. No.: US 2026/0176694 A1
(43) Pub. Date: Jun. 25, 2026

(54) METHODS FOR DETECTION OF CELL-FREE DNA (CFDNA) AND USES THEREOF FOR DIAGNOSING, TREATING, AND/OR MONITORING ALZHEIMER'S DISEASE

(71) Applicants: SEQ BIOMARQUE, LLC, Yorba Linda, CA (US); THE JOHNS HOPKINS UNIVERTSITY, Baltmore, MD (US)

(72) Inventors: Christopher A. Hamm, Chicago, IL (US); Victot E. Velculescu, Glenwood, MD (US); Akshaya Annapragade, Baltimore, MD (US); Sarah Bacus, Hindsdale, IL (US)

(21) Appl. No.: 19/127,002

(22) PCT Filed: Nov. 3, 2023
(86) PCT No.: PCT/US23/78701

§ 371 Date: May 2, 2025

Related U.S. Application Data
(60) Provisional application No. 63/422,364, filed on Nov. 3, 2022.

(51) Int. Cl. C12Q 1/6883

(52) U.S. Cl. CPC C12Q 1/6883; C12Q 2600/112

(57) ABSTRACT

Provided herein are biomarkers present in cell-free DNA (cfDNA) for the early detection of pre-clinical Alzheimer's Disease (AD), mild cognitive impairment (MCI), or AD in a subject. The detection of such biomarkers in a subject may be used to inform methods of treating a subject with a therapy (e.g., a drug or biologic) for pre-clinical Alzheimer's Disease (AD), mild cognitive impairment (MCI), or AD. The biomarkers disclosed herein may also be used in methods to monitor the progression of pre-clinical AD, MCI, or AD.

### FIELD

The present disclosure generally relates to biomarkers present in cell-free DNA (cfDNA) for the detection of pre-clinical Alzheimer's Disease (AD), mild cognitive impairment (MCI), or AD and uses thereof in methods of treatment and monitoring disease progression.

### BACKGROUND

Alzheimer's disease (AD) is a neurodegenerative disease of undefined etiology and limited diagnostic markers. Clinicopathological studies suggest that AD pathology (particularly the buildup of amyloid plaques) begins 10-20 years before cognitive symptoms. However, a definitive diagnosis of AD can still only be obtained via neuropathologic evaluation at autopsy. Data suggest an early and insidious pathogenesis of AD, the clinical manifestation of which becomes apparent only after substantial neuronal cell death and synapse loss has taken place. However, current treatment modalities are limited by imperfect diagnostic parameters and a complete inability to identify the early pathogenic disease process.

Early diagnosis is an essential step in the treatment of Alzheimer's Disease, as early diagnosis allows subjects to receive drugs that may slow disease progression. However, an accurate diagnosis can be difficult, especially for patients having mild or early-stage Alzheimer's Disease. An unambiguous diagnosis may be made by examining the pathology of brain tissue, but this is only feasible posthumously.

Accordingly, a need exists for diagnostic methods to detect biomarkers associated with Alzheimer's disease and the use of such biomarkers to identify and treat Alzheimer's Disease.

### SUMMARY

The present disclosure relates to methods for detecting and quantifying DNA including DNA fragments in cell-free DNA (cfDNA) in a biological sample (e.g., blood, serum, or cerebral spinal fluid (CSF)) and methods of using the same for diagnosing, treating, and/or monitoring the progression (e.g, advancement of disease severity) of a neurodegenerative disease or disorder (e.g., pre-clinical Alzheimer's Disease (AD), mild cognitive impairment (MCI), or AD). The variability in the amount and/or length of fragments in cfDNA obtained from a subject as compared to a normal subject (e.g., a subject without Alzheimer's Disease) may be used to provide early detection or diagnose pre-clinical Alzheimer's Disease (AD), mild cognitive impairment (MCI), or AD.

In an aspect, the present disclosure provides a method of treating a subject with an Alzheimer's Disease therapy, the method comprising: obtaining a biological sample comprising cfDNA from the subject; determining the length and amount of cell-free DNA (cfDNA) fragments in the biological sample; and treating the subject with the Alzheimer's Disease therapy if the amount of short cfDNA fragments is variable from (e.g, is less than or greater than) the amount of long cfDNA fragments.

In another aspect, the present disclosure provides a method of treating a subject with an Alzheimer's Disease therapy, the method comprising: obtaining a biological sample comprising cfDNA from the subject; determining a ratio of short cfDNA fragments to long cfDNA fragments in the biological sample; and treating the subject with the Alzheimer's Disease therapy if the ratio of short cfDNA fragments to long cfDNA fragments in the biological sample is variable from (e.g., is less than or greater than) a ratio of short cfDNA fragments to long cfDNA fragments in a reference sample.

In yet another aspect, the present disclosure provides a method of treating a subject with an Alzheimer's Disease therapy, the method comprising: obtaining a biological sample comprising cfDNA from the subject; determining the presence of aneuploidy in the cfDNA; and treating the subject with the Alzheimer's Disease therapy if the amount of aneuploidy in the cfDNA is variable from (e.g., is less than or greater than) the amount of aneuploidy in a reference sample.

In another aspect, the present disclosure provides a method of treating a subject with an Alzheimer's Disease therapy, the method comprising: obtaining a biological sample comprising cfDNA from the subject; determining the amount of transposable elements in the cfDNA; and treating the subject with the Alzheimer's Disease therapy if the amount of transposable elements in the cfDNA is variable from (e.g., is less than or greater than) the amount of transposable elements in a reference sample.

In yet another aspect, the present disclosure provides a method of treating a subject with an Alzheimer's Disease therapy, the method comprising: obtaining a biological sample comprising cfDNA from the subject; determining the presence of one or more transposable elements in the cfDNA; and treating the subject with the Alzheimer's Disease therapy if the one or more transposable elements are detected in the cfDNA.

In some embodiments, the subject has pre-clinical Alzheimer's Disease (AD), mild cognitive impairment (MCI), or AD.

In some embodiments, the reference sample is from a subject or subjects that does not have pre-clinical Alzheimer's Disease (AD), mild cognitive impairment (MCI), or AD.

In some embodiments, the biological sample is blood or plasma.

In some embodiments, the biological sample is cerebral spinal fluid.

In some embodiments, the short cfDNA fragments are about 100 to about 150 base pairs in length.

In some embodiments, the long cfDNA fragments are about 151 to about 220 base pairs in length.

In some embodiments, the transposable elements are selected from the group consisting of: BLACKJACK #DNA_hAT-Blackjack, Charlie18a #DNA_hAT-Charlie, Charlie25 #DNA_hAT-Charlie, Charlie29a #DNA_hAT-Charlie, Charlie7 #DNA hAT-Charlie, EuthAT-2 #DNA_hAT-Ac, Eutr15 #DNA_hAT-Tip100, Kangalla #DNA_TcMar-Tc2, MER104#DNA_TcMar-Tc2, MER124 #DNA, MER44A #DNA_TcMar-Tigger, MER44B #DNA_TcMar-Tigger, MER63C #DNA_hAT-Tip100, Tigger1 #DNA_TcMar-Tigger, Tigger10 #DNA_TcMar-Tigger, Tigger #13a #DNA_TcMar-Tigger, Tigger14a #DNA_TcMar-Tigger, Tigger15a #DNA_TcMar-Tigger, Tigger2 #DNA_TcMar-Tigger, Tigger22N1 #DNA_TcMar-Tigger, Tigger23a #DNA_TcMar-Tigger, Tigger2a #DNA_TcMar-Tigger, TIgget2b_Pri #DNA_TcMar-Tigger, Tigger3 #DNA_TcMar-Tigger, Tigger3a #DNA_TcMar-Tigger, Tigger3b #DNA_TcMar-Tigger, Tigger4 #DNA_TcMar-Tigger, and X9b_DNA #DNA_TcMar-Tigger.

In an aspect, the present disclosure provides a method for monitoring the progression of pre-clinical Alzheimer's Disease (AD), mild cognitive impairment (MCI), or AD in a subject in need thereof, the method comprising: obtaining a first biological sample from the subject at a first time; quantitating the length of cell-free DNA (cfDNA) fragments in the biological sample and/or a ratio of short cfDNA fragments to long cfDNA fragments in the first biological sample at the first time; obtaining a second biological sample from the subject at a second time; quantitating the length of cell-free DNA (cfDNA) fragments in the biological sample and/or a ratio of short cfDNA fragments to long cfDNA fragments in the second biological sample at the second time; and determining that the pre-clinical Alzheimer's Disease (AD), mild cognitive impairment (MCI), or AD has progressed where the length of cell-free DNA (cfDNA) fragments in the second biological sample is variable from (e.g, has increased or decreased) the length of cell-free DNA (cfDNA) fragments in the first biological sample or the ratio of short cfDNA fragments to long cfDNA fragments is variable from (e.g., has increased or decreased) the ratio of short cfDNA fragments to long cfDNA fragments in the first biological sample, wherein the first time precedes the second time.

In another aspect, the present disclosure provides a method for monitoring the progression of pre-clinical Alzheimer's Disease (AD), mild cognitive impairment (MCI), or AD in a subject in need thereof, the method comprising: obtaining a first biological sample from the subject at a first time; quantitating the amount of aneuploidy in the first biological sample at the first time; obtaining a second biological sample from the subject at a second time; quantitating the amount of aneuploidy in the second biological sample at the second time; and determining that the pre-clinical Alzheimer's Disease (AD), mild cognitive impairment (MCI), or AD has progressed where the amount of aneuploidy in the second biological sample is variable from (e.g., is lower or higher than) the amount of aneuploidy in the first biological sample, wherein the first time precedes the second time.

In another aspect, the disclosure provides a method for monitoring the progression of pre-clinical Alzheimer's Disease (AD), mild cognitive impairment (MCI), or AD in a subject in need thereof, the method comprising: obtaining a first biological sample from the subject at a first time; quantitating the amount of transposable elements in the first biological sample at the first time; obtaining a second biological sample from the subject at a second time; quantitating the amount of transposable elements in the second biological sample at the second time; and determining that the pre-clinical Alzheimer's Disease (AD), mild cognitive impairment (MCI), or AD has progressed where the amount of transposable elements in the second biological sample is variable from (e.g, is lower or higher than) the amount of transposable elements in the first biological sample, wherein the first time precedes the second time.

In yet another aspect, the disclosure provides a method of diagnosing a neurodegenerative disease or disorder in a subject, the method comprising: obtaining a biological sample from the subject; and determining the length of cell-free DNA (cfDNA) fragments in the biological sample, wherein the subject is diagnosed as having the neurodegenerative disease or disorder if the amount of short cfDNA fragments is variable from (e.g, is lower or higher than) the amount of long cfDNA fragments.

In another aspect, the disclosure provides a method of diagnosing a neurodegenerative disease or disorder in a subject, the method comprising: obtaining a biological sample from the subject; and determining the ratio of short cfDNA fragments to long cfDNA fragments in the biological sample, wherein the subject is diagnosed as having the neurodegenerative disease or disorder if the ratio of short cfDNA fragments to long cfDNA fragments in the biological sample is variable from (e.g, is lower or higher than) a ratio of short cfDNA fragments to long cfDNA fragments in a reference sample.

In some embodiments, the neurodegenerative disease or disorder is pre-clinical Alzheimer's Disease (AD), mild cognitive impairment (MCI), or AD.

In some embodiments, the biological sample is blood.

In some embodiments, the biological sample is cerebral spinal fluid.

The method of any one of claims 15-16, wherein the reference sample is from a subject that does not have pre-clinical Alzheimer's Disease (AD), mild cognitive impairment (MCI), or AD.

The present disclosure provides a method of treating a subject with a therapy for a neurodegenerative disease or disorder comprising the steps of: obtaining a biological sample from the subject, determining the length of cell-free DNA (cfDNA) fragments (e.g., fragments between 100 and 300 bases in length) in the biological sample, and treating the subject with the therapy for the neurodegenerative disease or disorder if the amount of short cfDNA fragments (e.g., fragments from 100 to 150 bases in length) is variable from (e.g, is lower or higher than) the amount of long cfDNA fragments (e.g., fragments from 151 to 220 base pairs in length) in the biological sample.

The present disclosure provides a method of treating a subject with a therapy for a neurodegenerative disease or disorder comprising the steps of: obtaining a biological sample from the subject, determining the length of cell-free DNA (cfDNA) fragments (e.g., fragments between 100 and 300 bases in length) in the biological sample, and treating the subject with the therapy for the neurodegenerative disease or disorder if the amount of short cfDNA fragments (e.g., fragments from 100 to 150 bases in length) is variable from (e.g, is lower or higher than) the amount of long cfDNA fragments (e.g., fragments from 151 to 220 base pairs in length) in the biological sample.

The present disclosure also provides a method of treating a subject with a therapy for Alzheimer's Disease comprising the steps of: obtaining a biological sample from the subject, determining the ratio of short cfDNA fragments (e.g., fragments from 100 to 150 bases in length) to long cfDNA fragments (e.g., fragments from 151 to 220 base pairs in length) in the biological sample; and treating the subject with the Alzheimer's Disease therapy if the ratio of short cfDNA fragments to long cfDNA fragments in the biological sample is variable from (e.g, is lower or higher than) the ratio of short cfDNA fragments to long cfDNA fragments in a reference sample.

The present disclosure further provides a method of treating a subject with a therapy for Alzheimer's Disease comprising the steps of: obtaining a biological sample from the subject, determining the ratio of short cfDNA fragments (e.g., fragments from 100 to 150 bases in length) to long cfDNA fragments (e.g., fragments from 151 to 220 base pairs in length) in the biological sample; and treating the subject with the Alzheimer's Disease therapy if the ratio of short cfDNA fragments to long cfDNA fragments in the biological sample is variable from (e.g, is lower or higher than) the ratio of short cfDNA fragments to long cfDNA fragments in a reference sample.

In some embodiments, the neurodegenerative disease or disorder is Alzheimer's Disease.

In some embodiments, the reference sample is from a subject that does not have Alzheimer's Disease.

In some embodiments, the cfDNA fragments are 100 to 300 base pairs in length.

In some embodiments, the short cfDNA fragments are 100 to 150 base pairs in length.

In some embodiments, the long cfDNA fragments are 151 to 220 base pairs in length.

In some embodiments, the biological sample is blood.

In some embodiments, the biological sample is cerebral spinal fluid.

The present disclosure also provides a method of diagnosing a neurodegenerative disease or disorder in a subject comprising the steps of: obtaining a biological sample from the subject, and determining the length of cell-free DNA (cfDNA) fragments in the biological sample or the ratio of small cfDNA fragments to large cfDNA fragments in the biological sample, wherein the subject is diagnosed as having the neurodegenerative disease or disorder if the amount of short cfDNA fragments (e.g., fragments from 100 to 150 bases in length) is variable from (e.g, is lower or higher than) the amount of long cfDNA fragments (e.g., fragments from 151 to 220 base pairs in length) in the biological sample.

The present disclosure also provides a method of diagnosing Alzheimer's Disease in a subject comprising the steps of: obtaining a biological sample from the subject, and determining the length of cell-free DNA (cfDNA) fragments in the biological sample or the ratio of small cfDNA fragments to large cfDNA fragments in the biological sample, wherein the subject is diagnosed as having Alzheimer's Disease if the amount of short cfDNA fragments (e.g., fragments from 100 to 150 bases in length) is variable from (e.g, is lower or higher than) the amount of long cfDNA fragments (e.g., fragments from 151 to 220 base pairs in length) in the biological sample.

The present disclosure also provides a method of diagnosing a neurodegenerative disease or disorder in a subject comprising the steps of: obtaining a biological sample from the subject, and determining the length of cell-free DNA (cfDNA) fragments in the biological sample or the ratio of small cfDNA fragments to large cfDNA fragments in the biological sample, wherein the subject is diagnosed as having the neurodegenerative disease or disorder if the ratio of short cfDNA fragments to long cfDNA fragments in the biological sample is variable from (e.g, is lower or higher than) the ratio of short cfDNA fragments to long cfDNA fragments in a reference sample.

The present disclosure also provides a method of diagnosing Alzheimer's Disease in a subject comprising the steps of: obtaining a biological sample from the subject, and determining the length of cell-free DNA (cfDNA) fragments in the biological sample or the ratio of small cfDNA fragments to large cfDNA fragments in the biological sample, wherein the subject is diagnosed as having Alzheimer's Disease if the ratio of short cfDNA fragments to long cfDNA fragments in the biological sample is variable from (e.g, is lower or higher than) the ratio of short cfDNA fragments to long cfDNA fragments in a reference sample.

In some embodiments, the neurodegenerative disease or disorder is Alzheimer's Disease.

In some embodiments, the reference sample is from a subject that does not have Alzheimer's Disease.

In some embodiments, the cfDNA fragments are 100 to 300 base pairs in length.

In some embodiments, the short cfDNA fragments are 100 to 150 base pairs in length.

In some embodiments, the long cfDNA fragments are 151 to 220 base pairs in length.

In some embodiments, the biological sample is blood.

In some embodiments, the biological sample is cerebral spinal fluid.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 shows extracted cfDNA from patient samples (normal liver sample, normal donor sample, and sample from a Alzheimer's Disease patient). Liver Normal are healthy controls from to provide a comparison to the other data sets and additional data for model training.

FIG. 2 shows that the fragmentation analysis of normal and AD samples identifies a greater presence of aneuploidy in the fragmentome of AD samples than for normal samples.

FIG. 3 shows the fragmentation profile for AD samples versus normal samples (liver and Normal Donors) plotted along each chromosome. The AD profile exhibits a marked difference between the AD samples and the two normal samples.

FIG. 4 demonstrates that 28 of 209 Transposable Elements show significantly greater representation in AD plasma than Normal plasma (p<0.05, after Bonferroni correction).

### DETAILED DESCRIPTION

The present disclosure provides biomarkers present in cell-free DNA (cfDNA) for the detection and/or determination of pre-clinical Alzheimer's Disease (AD), mild cognitive impairment (MCI), or AD in a subject. The detection of such biomarkers in a subject and their variability in amount and/or size (e.g, increase or decrease) as compared the same biomarkers present in a reference sample (or compared to a threshold) may be used to inform methods of treating a subject with a therapy (e.g., an effective amount of a therapy such as a drug or biologic). The biomarkers disclosed herein may also be used in methods to monitor the progression of pre-clinical AD, MCI, or AD.

Such methods may comprise obtaining a biological sample comprising cfDNA from a subject, determining the length of cell-free DNA (cfDNA) fragments in the biological sample or the ratio of short cfDNA fragments (e.g., ˜100-150 base pairs) to long cfDNA fragments (e.g., ˜151-220 base pairs) in the biological sample, and treating the subject with an Alzheimer's Disease therapy if the level (amount) of short cfDNA fragments or the ratio of short cfDNA fragments to long cfDNA fragments in the biological sample is variable from the amount of long cfDNA fragments or the ratio of short cfDNA fragments to long cfDNA fragments in a reference sample (e.g., a biological sample obtained from a subject without Alzheimer' Disease (AD). Additionally or alternatively, such methods may comprise obtaining a biological sample comprising cfDNA from a subject, determining the presence of aneuploidy in the cfDNA, and treating the subject with the Alzheimer's Disease therapy if the amount of aneuploidy in the biological sample is variable from the amount of aneuploidy detected in a reference sample (e.g., a biological sample obtained from a subject without Alzheimer's Disease). Additionally or alternatively, such methods may comprise obtaining a biological sample comprising cfDNA from a subject, determining the presence of transposable elements in the cfDNA, and treating the subject with the Alzheimer's Disease therapy if the amount of transposable elements in the biological sample is variable from the amount of transposable elements detected in a reference sample (e.g., a biological sample obtained from a subject without Alzheimer's Disease).

Surprisingly, the inventors have discovered that when neurons die in pre-clinical AD, mild cognitive impairment (MCI), or AD, neuronal DNA is released into the bloodstream (cfDNA) and the variability (e.g, increase or decrease) in the length and/or amount of DNA fragments is predictive of the occurrence of pre-clinical AD, mild cognitive impairment (MCI), or AD including its progression. The ability to detect the presence of pre-clinical AD, mild cognitive impairment (MCI), or AD will allow the detection of such disease or disorder in its early stages where treatment is more likely to be effective.

### Definitions

Prior to setting forth the invention in detail, definitions of certain terms to be used herein are provided. Unless defined otherwise, all technical and scientific terms used herein have the same meaning as is commonly understood by one of skill in the art.

As used herein, a “biological sample” is comprised of biologic material isolated from a subject and includes, without limitation, blood, serum, tissue, plasma or cerebrospinal fluid. Certain embodiments provide methods for collection of a sample (e.g., blood and other materials) useful for diagnostic purposes.

Where the term “comprising” is used in the present description and the claims, it does not exclude other elements or steps. For the purposes of the present invention, the term “consisting of” is considered to be a preferred embodiment of the term “comprising”.

The term “fragment” or “fragmentation” (e.g., a DNA fragment or fragmented DNA), as used herein, can refer to a portion of a polynucleotide or polypeptide sequence that comprises at least 3 consecutive nucleotides. A nucleic acid fragment can be double-stranded or single-stranded, methylated or unmethylated, intact or nicked, complexed or not complexed with other macromolecules, e.g., lipid particles, proteins.

As used herein, the term “amount” or “level” means quantity, number or concentration of a DNA fragment or DNA fragmentation.

As used herein, a DNA fragmentation level in an individual without a neurodegenerative disease or disorder includes, for example, a DNA fragmentation level in an individual who is representative of an average level of the DNA fragmentation in a population of individuals without the neurodegenerative disease or disorder. Thus, a level in an individual without the neurodegenerative disease or disorder may be determined by, for example, determining an average level of the DNA fragmentation in a population of individuals without the neurodegenerative disease or disorder.

As used herein, a DNA fragmentation level in an individual that is not at risk of developing the neurodegenerative disease or disorder includes, for example, a DNA fragmentation level in an individual who is representative of an average level of the DNA fragmentation in a population of individuals that are not at risk of developing the neurodegenerative disease or disorder. Thus, a level in an individual that is not at risk of developing the neurodegenerative disease or disorder may be determined by, for example, determining an average level of the DNA fragmentation in a population of individuals that are not at risk of developing the neurodegenerative disease or disorder.

As used herein, the term “subject” includes human and animals which are capable of suffering from or afflicted with dementia associated with a CNS disorder, including neurodegenerative diseases such as Alzheimer's Disease, or any disorder involving, directly or indirectly, Alzheimer's Disease. Examples of subjects include mammals, e.g., humans, non-human primates, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from Alzheimer's Disease or Alzheimer's Disease-associated dementia. In embodiments, the subject is a human between the ages of about 45-50, about 50 to about 55, about 55 to about 60, about 60 to about 65, about 65 to about 70, about 70 to about 75, about 75 to about 80, about 80 to about 85, about 85 to about 90, or greater than 90 years old.

As used herein, a “CDR” score means a score generated using the Clinical Dementia Rating assessment protocol developed at the Washington University Medical School (see Morris, C. J., Neurology, 1993; 43:2412-2414). A person determined to have no dementia (i.e. without detectable dementia) is designated a “0”; very mild dementia=0.5; mild dementia=1; moderate dementia=2; severe dementia=3. Individuals diagnosed with possible/probable dementia of the Alzheimer's type (DAT) are usually CDR 1 or greater. During early stages (CDR 0.5, often lasting 2-5 years or longer), the majority of individuals meet clinical criteria for mild cognitive impairment (MCI) (Peterson et al., Arch. Neurol, 1999; 56:303).

As used herein, a Mini-Mental State Examination (“MMSE”) score means a score generated using a MMSE protocol for evaluating cognitive therapy. As used herein, a patient with an MMSE score of 27-30 is considered to have no cognitive impairment, a patient with an MMSE score of 21-26 is considered to have mild cognitive impairment, a patient with an MMSE score of 11-20 is considered to have moderate cognitive impairment, and a patient with an MMSE score of 0 -10 is considered to have severe cognitive impairment.

A used herein, the term “variable” or “variability” refers to a lack of a consistent or fixed pattern between one data set and another data set (e.g., a data set obtained from a subject suspected of having Alzheimer's Disease as compared to a data set obtained from a subject without Alzheimer's Disease). Variability in data values may be reflected by an increase or a decrease including for example, a statistically significant increase or decrease, in the data values (e.g, an increase or a decrease in the mean or median of the data values) in one set versus the data values in a second set (e.g., a 5%, 10%, 15%, 20%, 25%, 30%, 35%, 40%, 45%, 50%, 55%, 60%, 65%, 70%, 75%, 80%, 90%, 95%, 100%, 150%, 200%, 250%, 300%, 350%, 400%, 450%, 500% or more difference in the mean or median between the two data sets). Data values that are dispersed or spread out between two data sets are considered variable.

### Detection and Quantification of DNA Fragmentation

Any method known in the art for detection and quantification of the amount of DNA fragmentation in a biological sample may be used in the methods disclosed herein. In certain embodiments, the DNA fragmentation pattern in a biological sample is detected using Next Generation Sequencing (NGS).

For example, whole genome sequencing (WGS) with or without machine learning may be performed to analyze DNA fragmentation patterns including, for example, length of cell-free DNA (cfDNA) in a biological sample. See, e.g., Cristiano et al. (2019) Nature 570(7761): 385-389. Such methods may use 5 Mb windows for evaluating cfDNA fragmentation patterns to provide >20,000 reads per window at 1-2×genome coverage. Within each window, the coverage and size distribution of cfDNA fragments can be examined in healthy (or normal) subjects and subjects having a neurodegenerative disease or disorder. The genome-wide pattern from a subject can be compared to reference (or normal/heathy) subjects to determine if the DNA fragmentation pattern is likely healthy (or normal) or indicative of the presence of neurodegenerative disease or disorder.

In an embodiment, the DNA fragment length (e.g., average or median overall DNA fragment length) in a subject predicted or diagnosed with a neurodegenerative disease or disorder is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides shorter than the DNA fragment length from a heathy or normal subject (e.g., a subject without the neurodegenerative disease or disorder). In another embodiment, the DNA fragment length (e.g., average or median overall DNA fragment length) in a subject predicted or diagnosed with a neurodegenerative disease or disorder is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides larger than the DNA fragment length from a heathy or normal subject (e.g., a subject without the neurodegenerative disease or disorder).

Alternatively, a fraction of small cfDNA fragments (e.g., 100 to 150 bp) to larger cfDNA fragments (151 to 220 bp) may be determined for one or more windows of 1 Mb to 10 Mb including more than one window covering approximately 0.5 to 3.0 Gb of the genome. A subject is predicted to have a neurodegenerative disease or disorder or is diagnosed with the disease or disorder if a fraction of small cfDNA fragments to large cfDNA fragments is different (e.g., larger or smaller) than the fraction of small cfDNA fragments to large cfDNA fragments from a heathy or normal subject (e.g., a subject without the neurodegenerative disease or disorder).

Alternatively, the level of DNA fragmentation is determined by electrophoresis. For example, in some embodiments, the level of DNA fragmentation is determined by, without limitation, pulsed field electrophoresis (PFGE), one-or two-dimensional electrophoresis, or capillary electrophoresis. Those skilled in the art will recognize still further quantitative electrophoresis methods suitable for practicing the present disclosure.

In certain embodiments, the level of DNA fragmentation in a biological is detected using mass spectrometry. In particular embodiments, the level of DNA fragmentation is detected using selected reaction monitoring mass spectrometry (SRM-MS). In other embodiments, the level of DNA fragmentation in the sample is detected using other quantitative mass spectrometry techniques, including, without limitation, spectral counting, isobaric mass tagging, or ion mobility mass spectrometry.

### Diagnosis of a Neurodegenerative Disease or Disorder

The present disclosure also provides a method of diagnosing a neurodegenerative disease or disorder form a biological sample (e.g, a blood, serum, or CSF sample) obtained from a subject. Such methods may comprises obtaining a biological sample (e.g., a blood sample or CSF) from the subject, and determining the length of cell-free DNA (cfDNA) fragments in the biological sample or the ratio of short cfDNA fragments to long cfDNA fragments in the biological sample.

The subject may be diagnosed as having the neurodegenerative disease or disorder if the level of short cfDNA fragments in the biological sample is greater than the level of long cfDNA fragments in the biological sample. Alternatively, the subject may be diagnosed as having the neurodegenerative disease or disorder if the level of short cfDNA fragments in the biological sample is less than the level of long cfDNA fragments in the biological sample. In an embodiment, the cfDNA fragments in the biological sample may be about 100 to about 300 base pairs in length including, for example, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, or about 300 base pairs in length.

Alternatively, the subject may be diagnosed as having the neurodegenerative disease or disorder if the ratio of short cfDNA fragments to long cfDNA fragments in the biological sample is greater than the ratio of short cfDNA fragments to long cfDNA fragments in a reference or control sample. In another embodiment, the subject may be diagnosed as having the neurodegenerative disease or disorder if the ratio of short cfDNA fragments to long cfDNA fragments in the biological sample is less than the ratio of short cfDNA fragments to long cfDNA fragments in a reference or control sample.

In an embodiment, the short cfDNA fragments may be about 100 to about 150 base pairs in length and/or the long cfDNA fragments may be about 151 to about 220 base pairs in length.

The present disclosure also provides a method of treating a subject with an Alzheimer's Disease therapy, the method comprising: obtaining a biological sample comprising cfDNA from the subject; determining the length and amount of cell-free DNA (cfDNA) fragments in the biological sample; and treating the subject with the Alzheimer's Disease therapy if the amount of short cfDNA fragments differs from (e.g, is less than or greater than) the amount of long cfDNA fragments.

The present disclosure also provides a method of treating a subject with an Alzheimer's Disease therapy, the method comprising: obtaining a biological sample comprising cfDNA from the subject; determining a ratio of short cfDNA fragments to long cfDNA fragments in the biological sample; and treating the subject with the Alzheimer's Disease therapy if the ratio of short cfDNA fragments to long cfDNA fragments in the biological sample differs from (e.g., is less than or greater than) a ratio of short cfDNA fragments to long cfDNA fragments in a reference sample.

The present disclosure also provides a method of treating a subject with an Alzheimer's Disease therapy, the method comprising: obtaining a biological sample comprising cfDNA from the subject; determining the presence of aneuploidy in the cfDNA; and treating the subject with the Alzheimer's Disease therapy if the level of aneuploidy in the cfDNA differs from (e.g., is less than or greater than) the level of aneuploidy in a reference sample.

The present disclosure also provides a method of treating a subject with an Alzheimer's Disease therapy, the method comprising: obtaining a biological sample comprising cfDNA from the subject; determining the amount of transposable elements in the cfDNA; and treating the subject with the Alzheimer's Disease therapy if the amount of transposable elements in the cfDNA differs from (e.g., is less than or greater than) the amount of transposable elements in a reference sample.

The transposable elements may include one or more elements selected from the group consisting of: BLACKJACK #DNA_hAT-Blackjack, Charlie18a #DNA_hAT-Charlie, Charlie25 #DNA_hAT-Charlie, Charlie29a #DNA_hAT-Charlie, Charlie7 #DNA_hAT-Charlie, EuthAT-2 #DNA_hAT-Ac, Eutr15 #DNA_hAT-Tip100, Kangalla #DNA_TcMar-Tc2, MER104#DNA_TcMar-Tc2, MER124 #DNA, MER44A #DNA_TcMar-Tigger, MER44B #DNA_TcMar-Tigger, MER63C #DNA_hAT-Tip100, Tigger1 #DNA_TcMar-Tigger, Tigger10 #DNA_TcMar-Tigger, Tigger #13a #DNA_TcMar-Tigger, Tigger14a #DNA_TcMar-Tigger, Tigger15a #DNA_TcMar-Tigger, Tigger2 #DNA_TcMar-Tigger, Tigger22N1 #DNA_TcMar-Tigger, Tigger23a #DNA_TcMar-Tigger, Tigger2a #DNA_TcMar-Tigger, TIgget2b_Pri #DNA_TcMar-Tigger, Tigger3 #DNA_TcMar-Tigger, Tigger3a #DNA_TcMar-Tigger, Tigger3b #DNA_TcMar-Tigger, Tigger4 #DNA_TcMar-Tigger, and X9b DNA #DNA_TcMar-Tigger.

The present disclosure also provides a method for monitoring the progression of pre-clinical Alzheimer's Disease (AD), mild cognitive impairment (MCI), or AD in a subject in need thereof, the method comprising: obtaining a first biological sample from the subject at a first time; quantitating the length of cell-free DNA (cfDNA) fragments in the biological sample and/or a ratio of short cfDNA fragments to long cfDNA fragments in the first biological sample at the first time; obtaining a second biological sample from the subject at a second time; quantitating the length of cell-free DNA (cfDNA) fragments in the biological sample and/or a ratio of short cfDNA fragments to long cfDNA fragments in the second biological sample at the second time; and determining that the pre-clinical Alzheimer's Disease (AD), mild cognitive impairment (MCI), or AD has progressed where the length of cell-free DNA (cfDNA) fragments in the second biological sample differs from (e.g, has increased or decreased) as compared to the length of cell-free DNA (cfDNA) fragments in the first biological sample or the ratio of short cfDNA fragments to long cfDNA fragments differs from (e.g., has increased or decreased) in the second biological sample as compared to the ratio of short cfDNA fragments to long cfDNA fragments in the first biological sample, wherein the first time precedes the second time.

The present disclosure provides a method for monitoring the progression of pre-clinical Alzheimer's Disease (AD), mild cognitive impairment (MCI), or AD in a subject in need thereof, the method comprising: obtaining a first biological sample from the subject at a first time; quantitating the amount of aneuploidy in the first biological sample at the first time; obtaining a second biological sample from the subject at a second time; quantitating the amount of aneuploidy in the second biological sample at the second time; and determining that the pre-clinical Alzheimer's Disease (AD), mild cognitive impairment (MCI), or AD has progressed where the amount of aneuploidy in the second biological sample differs from (e.g., is lower or higher than) the amount of aneuploidy in the first biological sample, wherein the first time precedes the second time.

The present disclosure also provides a method for monitoring the progression of pre-clinical Alzheimer's Disease (AD), mild cognitive impairment (MCI), or AD in a subject in need thereof, the method comprising: obtaining a first biological sample from the subject at a first time; quantitating the amount of transposable elements in the first biological sample at the first time; obtaining a second biological sample from the subject at a second time; quantitating the amount of transposable elements in the second biological sample at the second time; and determining that the pre-clinical Alzheimer's Disease (AD), mild cognitive impairment (MCI), or AD has progressed where the amount of transposable elements in the second biological sample differs from (e.g, is lower or higher than) the level of transposable elements in the first biological sample, wherein the first time precedes the second time.

The present disclosure also provides a method of diagnosing a neurodegenerative disease or disorder in a subject, the method comprising: obtaining a biological sample from the subject; and determining the length of cell-free DNA (cfDNA) fragments in the biological sample, wherein the subject is diagnosed as having the neurodegenerative disease or disorder if the amount of short cfDNA fragments differs from the amount of long cfDNA fragments.

The present disclosure also provides a method of diagnosing a neurodegenerative disease or disorder in a subject, the method comprising: obtaining a biological sample from the subject; and determining the ratio of short cfDNA fragments to long cfDNA fragments in the biological sample, wherein the subject is diagnosed as having the neurodegenerative disease or disorder if the ratio of short cfDNA fragments to long cfDNA fragments in the biological sample differs from a ratio of short cfDNA fragments to long cfDNA fragments in a reference sample. The reference sample may be from a subject that does not have pre-clinical Alzheimer's Disease (AD), mild cognitive impairment (MCI), or AD.

In other embodiments, a normal level (or reference level) of short and/or long DNA fragments can be an average level of short or long DNA fragments in samples of one or more healthy subjects having a Mini-Mental State Examination (“MMSE”) score between 27 and 30, such as subjects in the same age group and, optionally, of the same gender. In alternative embodiments, a normal ratio (or reference ratio) of short to long DNA fragments can be an average ratio of short to long DNA fragments in samples of one or more healthy subjects having a Mini-Mental State Examination (“MMSE”) score between 27 and 30, such as subjects in the same age group and, optionally, of the same gender. During the MMSE, a physician or other medical professional asks a patient a series of questions that are designed to test a range of everyday mental skills. Questions commonly asked include, for example, remembering and repeating the names of three common objects, stating the year, date, season, and day of the week, counting backwards from 100 in increments of 7, spelling the word “world” backwards, naming familiar objects as the examiner points to them, identifying the location of the examiner's office, repeating a common phrase after it is stated by the Examiner, copying a picture of two interlocking shapes, and following a three-part series of instructions (e.g., pick up a piece of paper, fold it in half, and place it on the floor). The maximum score on the MMSE examination is 30 points. In general, a patient with an MMSE score of 27-30 is considered to have no cognitive impairment, a patient with an MMSE score of 21-26 is considered to have mild cognitive impairment, a patient with an MMSE score of 11-20 is considered to have moderate cognitive impairment, and a patient with an MMSE score of 0-10 is considered to have severe cognitive impairment. In certain embodiments, a patient with an MMSE score of 0-16 is considered to have advanced (moderately severe to severe) Alzheimer's disease.

A reference standard serves as a reference level for comparison, such that a sample from a subject can be compared to the reference standard to infer the Alzheimer's Disease status of the subject. A reference standard may be representative of the level of short and/or long DNA fragments (or their ratio) in a known subject, e.g., a subject known to be a normal subject, or a subject known to have a neurodegenerative disease or disorder.

Likewise, a reference standard may be representative of the level of short and/or long DNA fragments (or their ratio) in a population of known subjects, e.g., a population of subjects known to be normal subjects, or a population of subjects known to have a neurodegenerative disease or disorder. The reference standard may be obtained, for example, by pooling samples from a plurality of individuals and determining the level of short and/or long DNA fragments (or their ratio) in the pooled samples, to thereby produce a standard over an averaged population. Such a reference standard represents an average level of short and/or long DNA fragments (or their ratio) among a population of individuals.

A reference standard for short and/or long DNA fragments may also be obtained, for example, by averaging the level of short DNA fragments and long DNA fragments in individual samples obtained from a plurality of individuals without a neurodegenerative disease or disorder.

A reference standard may also be a collection of values each representing the level of DNA fragmentation in a known subject in a population of individuals. In certain embodiments, biological samples may be compared against such a collection of values to infer the neurodegenerative disease or disorder status of a subject.

In certain embodiments, the reference standard is an absolute value. In such embodiments, biological samples may be compared against the absolute value in order to infer the if a subject has a neurodegenerative disease or disorder. In a one embodiment, a comparison between the DNA fragmentation in a sample relative to a suitable control is made by executing a software classification algorithm. The skilled person can readily envision additional suitable controls that may be appropriate depending on the assay in question.

A subject having (e.g., a biological sample obtained from the subject having) a level of short cfDNA fragments that is greater than the level of long cfDNA fragments as compared to a normal subject (e.g., a subject without Alzheimer's Disease) may have Alzheimer's Disease, including early-stage Alzheimer's Disease, moderate or mid-stage Alzheimer's Disease, or severe or late-stage Alzheimer's Disease. In one embodiment, the level of short cfDNA fragments being greater than the level of long cfDNA fragments in a biological sample may be used to diagnose Alzheimer's disease in a subject having symptoms characteristic of early-stage Alzheimer's Disease, also known as prodromal Alzheimer's Disease.

A subject having (e.g., a biological sample obtained from the subject having) a level of short cfDNA fragments that is less than the level of long cfDNA fragments as compared to a normal subject (e.g., a subject without Alzheimer's Disease) may have Alzheimer's Disease, including early-stage Alzheimer's Disease, moderate or mid-stage Alzheimer's Disease, or severe or late-stage Alzheimer's Disease. In one embodiment, the level of short cfDNA fragments being less than the level of long cfDNA fragments in a biological sample may be used to diagnose Alzheimer's disease in a subject having symptoms characteristic of early-stage Alzheimer's Disease, also known as prodromal Alzheimer's Disease.

Alternatively, a subject having (e.g., a biological sample obtained from the subject having) a ratio of short cfDNA fragments to long cfDNA fragments greater than the ratio of short cfDNA fragments to long cfDNA fragments in a reference sample (e.g., a subject without Alzheimer's Disease) may have Alzheimer's Disease, including early-stage Alzheimer's Disease, moderate or mid-stage Alzheimer's Disease, or severe or late-stage Alzheimer's Disease. In one embodiment, the ratio of short cfDNA fragments to long cfDNA fragments in the biological sample being greater than the ratio of short cfDNA fragments to long cfDNA fragments in a reference sample may be used to diagnose Alzheimer's disease in a subject having symptoms characteristic of early-stage Alzheimer's Disease, also known as prodromal Alzheimer's Disease.

Additionally, a subject having (e.g., a biological sample obtained from the subject having) a ratio of short cfDNA fragments to long cfDNA fragments less than the ratio of short cfDNA fragments to long cfDNA fragments in a reference sample (e.g., a subject without Alzheimer's Disease) may have Alzheimer's Disease, including early-stage Alzheimer's Disease, moderate or mid-stage Alzheimer's Disease, or severe or late-stage Alzheimer's Disease. In one embodiment, the ratio of short cfDNA fragments to long cfDNA fragments in the biological sample being less than the ratio of short cfDNA fragments to long cfDNA fragments in a reference sample may be used to diagnose Alzheimer's disease in a subject having symptoms characteristic of early-stage Alzheimer's Disease, also known as prodromal Alzheimer's Disease.

In another embodiment, the level of short and long DNA fragments (or their ratio) may be used to diagnose Alzheimer's Disease in a subject having symptoms characteristic of “moderately severe cognitive decline,” also referred to as “moderate” or “mid-stage” Alzheimer's disease. Moderately severe cognitive decline is characterized by major gaps in memory and the emergence of deficits in cognitive function. At this stage, some assistance with day-to-day activities is indicated.

In another embodiment, the level of short and/or long DNA fragments (or their ratio) may be used to diagnose Alzheimer's Disease in a subject having symptoms characteristic of “severe cognitive decline,” also referred to as “moderate” or “mid-stage” Alzheimer's disease. In severe cognitive decline, memory difficulties continue to worsen, significant personality changes may emerge, and affected individuals typically need extensive help with customary daily activities.

In some embodiments, the level of short and long DNA fragments, as well as their ratio, in a biological sample obtained from a subject is compared to the normal level of short and long DNA fragments (or their ratio) in a subject, or subjects, without a neurodegenerative disease (e.g., Alzheimer's Disease), not at risk of developing the neurodegenerative disease or disorder, or to a reference standard. The normal level short and/or long DNA fragments (as well as their ratio) in a biological sample, or reference standard, can be an average level of short and/or long DNA fragments (or their ratio) in samples of one or more healthy subjects (e.g., subjects with a CDR score of 0), such as subjects in the same age group and, optionally, of the same gender and/or ethnicity.

Typically, the reference standard reference is a threshold value or a cut-off value. A “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of the DNA fragmentation level in properly banked historical subject samples may be used in establishing the predetermined corresponding reference value. In some embodiments, the predetermined corresponding reference value is the median measured level in a population of subjects including, for example, a population of subjects without the neurodegenerative disease or disorder.

### Methods for the Treatment or Prevention of Neurodegenerative Diseases or Disorders

The present disclosure provides methods for the treatment or prevention of a neurodegenerative disease or disorder such as pre-clinical Alzheimer's Disease (AD), mild cognitive impairment (MCI), or AD in a subject in need thereof. Such methods may comprise obtaining a biological sample comprising cfDNA from the subject, determining the variability in the length and/or amount of DNA fragments in cfDNA from the biological sample as compared to a reference standard (or threshold), and treating the subject with the Alzheimer's Disease therapy if there is variability in the length and/or amount of DNA fragments in cfDNA in the biological sample as compared to the reference standard (or threshold). A patient may be treated with an Alzheimer's Disease therapy if there is any variability of the length, amount, or type of any of the biomarkers disclosed herein as compared to a reference standard (or threshold).

Additionally, the present disclosure provides methods for the treatment or prevention of a neurodegenerative disease or disorder such as Alzheimer's Disease in a subject in need thereof comprising obtaining a biological sample from the subject, determining the ratio of short cfDNA fragments (e.g., fragments from 100 to 150 bases in length) to long cfDNA fragments (e.g., fragments from 151 to 220 base pairs in length)in the biological sample, and treating the subject with the Alzheimer's Disease therapy if the ratio of short cfDNA fragments to long cfDNA fragments in the biological sample is greater than the ratio of short cfDNA fragments to long cfDNA fragments in a reference sample.

As used herein, an Alzheimer's treatment comprises prescribing or administering one or more therapeutic interventions to slow, prevent, reverse, or change disease progression. An Alzheimer's treatment may be a drug or non-drug treatment. In some embodiments, an Alzheimer's treatment, or therapeutic, may treat one or more symptoms of disease. For example, a therapeutic intervention may comprise administering a therapeutically effective amount of at least one Alzheimer's therapeutic drug to the subject. In certain embodiments, the Alzheimer's therapeutic may be Razadyne® (galantamine), Exelon® (rivastigmine), Aricept® (donepezil), Namenda® (memantine), or a pharmaceutically acceptable salt or ester thereof. In a particular embodiment, an Alzheimer's treatment according to the disclosure comprises administering the therapeutic Aducanumab (Aduhelm™). In some embodiments, an Alzheimer's treatment comprises treatment with Suvorexant (Belsomra®), Citalopram (Celexa®), Fluoxetine (Prozac®), Paroxeine (Paxil®), Sertraline (Zoloft®), Trazodone (Desyrel®), Lorazepam (Ativan®), Oxazepam (Serax®), Aripiprazole (Abilify®), Clozapine (Clozaril®), Haloperidol (Haldol®), Olanzapine (Zyprexa®), Quetiapine (Seroquel®), Risperidone (Risperdal®), Ziprasidone (Geodon®), or Carbamazepine (Tegretol®). In other embodiments, an Alzheimer's treatment may comprise a non-drug therapeutic regimen, such as a behavioral therapy regimen.

The Alzheimer's therapeutics may be administered to a subject using a pharmaceutical composition. Suitable pharmaceutical compositions comprise an Alzheimer's therapeutic (or a pharmaceutically acceptable salt or ester thereof), and optionally comprise a pharmaceutically acceptable carrier, such as a pharmaceutical composition comprising galantamine, rivastigmine, donepezil or a pharmaceutically acceptable salt or ester of any of the foregoing (e.g., galantamine hydrobromide, rivastigmine tartrate, donepezil hydrochloride). In certain embodiments, these compositions optionally further comprise one or more additional therapeutic agents.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66:1-19 (1977), incorporated herein by reference. The salts can be prepared in situ during the final isolation and purification of the compounds of the invention, or separately by reacting a free base or free acid function with a suitable reagent. For example, a free base function can be reacted with a suitable acid. Furthermore, where the compounds carry an acidic moiety, suitable pharmaceutically acceptable salts thereof may, include metal salts such as alkali metal salts, e.g. sodium or potassium salts; and alkaline earth metal salts, e.g. calcium or magnesium salts.

The term “pharmaceutically acceptable ester”, as used herein, refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. As described above, the pharmaceutical compositions may additionally comprise a pharmaceutically acceptable carrier. The term carrier includes any and all solvents, diluents, or other liquid vehicle, dispersion or suspension aids, surface active agents, isotonic agents, thickening or emulsifying agents, preservatives, solid binders, lubricants and the like, suitable for preparing the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogen free water; isotonic saline; Ringer's solution; ethyl alcohol, and phosphate buffer solutions, as well as other nontoxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The terms “treat” or “treating” are used herein to mean to relieve, reduce or alleviate at least one symptom of a disease in a subject. For example, in relation to Alzheimer's Disease, the term “treat” includes relieving, reducing, or alleviating cognitive impairment (such as impairment of memory and/or orientation) or impairment of global functioning (overall functioning, including activities of daily living) and/or slowing down or reversing the progressive deterioration in global or cognitive impairment. Accordingly, the term “treat” also encompasses delaying or preventing onset prior to clinical manifestation of a disease or symptom of a disease and/or reducing the risk of developing or worsening of a symptom of a disease.

In some embodiments, administration of treatment for the neurodegenerative disease or disorder is followed by monitoring of the level of DNA fragmentation in a biological sample obtained from the subject and, optionally, comparing the level of DNA fragmentation to a normal level of DNA fragmentation. In some embodiments, administration of a first dose of a treatment for the neurodegenerative disease or disorder is followed by determining the level of DNA fragmentation, and if the level of DNA fragmentation is increased over the normal level, administering a second treatment for the neurodegenerative disease or disorder at a higher dose (e.g., 1.25, 1.5, 1.75, 2, 2.5, 3, 4, 5, 6, 7, 8, 9, or 10 times higher) than the first dose.

### Kits

In another aspect, the present disclosure provides kits for diagnosing a neurodegenerative disease or (e.g., Alzheimer's Disease) in a subject, which kits are useful for determining the level of DNA fragmentation in a biological sample obtained from the subject.

Kits may include materials and reagents adapted to selectively detect the presence of DNA fragmentation diagnostic for a neurodegenerative disease or disorder such as Alzheimer's Disease in a sample derived from a subject. For example, the kit may include reagents useful for performing an assay to detect DNA fragmentation.

In a further embodiment, the kit may contain instructions for suitable operational parameters in the form of a label or product insert. For example, the instructions may include information or directions regarding how to collect a sample, how to determine the level of DNA fragmentation in a sample, or how to correlate the level of DNA fragmentation in a sample with the Alzheimer's Disease status of a subject.

In another embodiment, the kit can contain one or more containers with DNA fragmentation samples, to be used as reference standards, suitable controls, or for calibration of an assay to detect DNA fragmentation in a test sample.

Computer systems are also provided having one or more processors and memory storing one or more programs for execution by the one or more processors. Such a system includes memory-storing instructions for causing the computer system to perform any of the methods described herein including a portion of any of the methods disclosed herein.

Features of the embodiments described herein can be implemented in, using, or with the assistance of a computer program product, such as a storage medium (media) or computer readable storage medium (media) having instructions stored thereon/in which can be used to program a processing system to perform any of the features presented herein. The storage medium (e.g., the memory) can include, but is not limited to, high-speed random access memory, such as DRAM, SRAM, DDR RAM or other random access solid state memory devices, and may include non-volatile memory, such as one or more magnetic disk storage devices, optical disk storage devices, flash memory devices, or other non-volatile solid state storage devices. In some embodiments, the memory may include one or more storage devices remotely located from the CPU(s). The memory, or alternatively the non-volatile memory device(s) within these memories, comprises a non-transitory computer readable storage medium.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1: A method of treating a subject with an Alzheimer's Disease therapy, the method comprising: obtaining a biological sample comprising cfDNA from the subject; determining the length and amount of cell-free DNA (cfDNA) fragments in the biological sample; and treating the subject with the Alzheimer's Disease therapy if the amount of short cfDNA fragments is variable from the amount of long cfDNA fragments.
Clause 2: A method of treating a subject with an Alzheimer's Disease therapy, the method comprising: obtaining a biological sample comprising cfDNA from the subject; determining a ratio of short cfDNA fragments to long cfDNA fragments in the biological sample; and treating the subject with the Alzheimer's Disease therapy if the ratio of short cfDNA fragments to long cfDNA fragments in the biological sample is variable from a ratio of short cfDNA fragments to long cfDNA fragments in a reference sample.
Clause 3: A method of treating a subject with an Alzheimer's Disease therapy, the method comprising: obtaining a biological sample comprising cfDNA from the subject; determining the presence of aneuploidy in the cfDNA; and treating the subject with the Alzheimer's Disease therapy if the amount of aneuploidy in the cfDNA is variable from the amount of aneuploidy in a reference sample.
Clause 4: A method of treating a subject with an Alzheimer's Disease therapy, the method comprising: obtaining a biological sample comprising cfDNA from the subject; determining the amount of transposable elements in the cfDNA; and treating the subject with the Alzheimer's Disease therapy if the amount of transposable elements in the cfDNA is variable from the amount of transposable elements in a reference sample.
Clause 5: The method of any one of Clauses 1-4, wherein the subject has pre-clinical Alzheimer's Disease (AD), mild cognitive impairment (MCI), or AD.
Clause 6: The method of any one of Clauses 2-4, wherein the reference sample is from a subject or subjects that does not have pre-clinical Alzheimer's Disease (AD), mild cognitive impairment (MCI), or AD.
Clause 7: The method of any one of Clauses 1-4, wherein the biological sample is blood or plasma.
Clause 8: The method of any one of Clauses 1-4, wherein the biological sample is cerebral spinal fluid.
Clause 9: The method of any one of Clauses 1-2, wherein the short cfDNA fragments are about 100 to about 150 base pairs in length.
Clause 10: The method of any one of Clauses 1-2, wherein the long cfDNA fragments are about 151 to about 220 base pairs in length.
Clause 11: The method of Clause 4, wherein the transposable elements are selected from the group consisting of: BLACKJACK #DNA_hAT-Blackjack, Charlie18a #DNA_hAT-Charlie, Charlie25 #DNA_hAT-Charlie, Charlie29a #DNA_hAT-Charlie, Charlie7 #DNA_hAT-Charlie, EuthAT-2 #DNA_hAT-Ac, Eutr15 #DNA_hAT-Tip100, Kangalla #DNA_TcMar-Tc2, MER104#DNA_TcMar-Tc2, MER124 #DNA, MER44A #DNA_TcMar-Tigger, MER44B #DNA_TcMar-Tigger, MER63C #DNA_hAT-Tip100, Tigger1 #DNA_TcMar-Tigger, Tigger10 #DNA_TcMar-Tigger, Tigger #13a #DNA_TcMar-Tigger, Tigger14a #DNA_TcMar-Tigger, Tigger15a #DNA_TcMar-Tigger, Tigger2 #DNA_TcMar-Tigger, Tigger22N1 #DNA_TcMar-Tigger, Tigger23a #DNA_TcMar-Tigger, Tigger2a #DNA_TcMar-Tigger, TIgget2b_Pri #DNA_TcMar-Tigger, Tigger3 #DNA_TcMar-Tigger, Tigger3a #DNA_TcMar-Tigger, Tigger3b #DNA_TcMar-Tigger, Tigger4 #DNA_TcMar-Tigger, and X9b DNA #DNA_TcMar-Tigger.
Clause 12: A method for monitoring the progression of pre-clinical Alzheimer's Disease (AD), mild cognitive impairment (MCI), or AD in a subject in need thereof, the method comprising: obtaining a first biological sample from the subject at a first time; quantitating the length of cell-free DNA (cfDNA) fragments in the biological sample and/or a ratio of short cfDNA fragments to long cfDNA fragments in the first biological sample at the first time; obtaining a second biological sample from the subject at a second time; quantitating the length of cell-free DNA (cfDNA) fragments in the biological sample and/or a ratio of short cfDNA fragments to long cfDNA fragments in the second biological sample at the second time; and determining that the pre-clinical Alzheimer's Disease (AD), mild cognitive impairment (MCI), or AD has progressed where the length of cell-free DNA (cfDNA) fragments in the second biological sample is variable from the length of cell-free DNA (cfDNA) fragments in the first biological sample or the ratio of short cfDNA fragments to long cfDNA fragments in the second biological sample is variable from the ratio of short cfDNA fragments to long cfDNA fragments in the first biological sample, wherein the first time precedes the second time.
Clause 13: A method for monitoring the progression of pre-clinical Alzheimer's Disease (AD), mild cognitive impairment (MCI), or AD in a subject in need thereof, the method comprising: obtaining a first biological sample from the subject at a first time; quantitating the amount of aneuploidy in the first biological sample at the first time; obtaining a second biological sample from the subject at a second time; quantitating the amount of aneuploidy in the second biological sample at the second time; and determining that the pre-clinical Alzheimer's Disease (AD), mild cognitive impairment (MCI), or AD has progressed where the amount of aneuploidy in the second biological sample is variable from the amount of aneuploidy in the first biological sample, wherein the first time precedes the second time.
Clause 14: A method for monitoring the progression of pre-clinical Alzheimer's Disease (AD), mild cognitive impairment (MCI), or AD in a subject in need thereof, the method comprising: obtaining a first biological sample from the subject at a first time; quantitating the amount of transposable elements in the first biological sample at the first time; obtaining a second biological sample from the subject at a second time; quantitating the amount of transposable elements in the second biological sample at the second time; and determining that the pre-clinical Alzheimer's Disease (AD), mild cognitive impairment (MCI), or AD has progressed where the amount of transposable elements in the second biological sample is variable from the level of transposable elements in the first biological sample, wherein the first time precedes the second time.
Clause 15: A method of diagnosing a neurodegenerative disease or disorder in a subject, the method comprising: obtaining a biological sample from the subject; and determining the length of cell-free DNA (cfDNA) fragments in the biological sample, wherein the subject is diagnosed as having the neurodegenerative disease or disorder if the amount of short cfDNA fragments is variable from the amount of long cfDNA fragments.
Clause 16: A method of diagnosing a neurodegenerative disease or disorder in a subject, the method comprising: obtaining a biological sample from the subject; and determining the ratio of short cfDNA fragments to long cfDNA fragments in the biological sample, wherein the subject is diagnosed as having the neurodegenerative disease or disorder if the ratio of short cfDNA fragments to long cfDNA fragments in the biological sample is variable from a ratio of short cfDNA fragments to long cfDNA fragments in a reference sample.
Clause 17: The method of any one of Clauses 15-16, wherein the neurodegenerative disease or disorder is pre-clinical Alzheimer's Disease (AD), mild cognitive impairment (MCI), or AD,
Clause 18: The method of any one of Clauses 15-16, wherein the biological sample is blood.
Clause 19: The method of any one of Clauses 15-16, wherein the biological sample is cerebral spinal fluid.
Clause 20: The method of any one of Clauses 15-16, wherein the reference sample is from a subject that does not have pre-clinical Alzheimer's Disease (AD), mild cognitive impairment (MCI), or AD.

This disclosure is further illustrated by the following examples which are provided to facilitate the practice of the disclosed methods. These examples do not limit the scope of the disclosure in any way.

### EXAMPLES

### Example 1: Detection of DNA Fragmentation and Aneuploidy in Human Plasma

195 plasma samples from Proteogenex, Inc. were selected for the study. The samples consisted of 95 Alzheimer's Disease patients and 100 normal donors. The following criteria were used to classify AD from normal samples:
Normal samples: i) no neurological deterioration observed in medical examinations, and/or ii) clinical diagnosis was normal.
Alzheimer's samples: i) MMSE 17 or lower; ii) verification by computerized tomography (CT) scan, iii) electroencephalogram (EEG) or magnetic resonance imaging (MRI); and/or iv) clinical diagnosis of Alzheimer's Disease.

DNA was isolated from plasma using the Qiagen Circulating Nucleic Acids Kit (Qiagen GmbH) and eluted in LoBind tubes (Eppendorf AG). Concentration and quality of cfDNA were assessed using the Bioanalyzer 2100 (Agilent Technologies).

NGS cfDNA libraries were prepared for whole genome sequencing using 5 to 250 ng of cfDNA as previously described (e.g., Phallen J. et al. Direct detection of early-stage cancers using circulating tumor DNA. Sci Transl Med 9, doi:10.1126/scitranslmed.aan2415 (2017)).

Genomic libraries were prepared using the standard NEBNext DNA Library Prep Kit for Illumina (New England Biolabs) with the following modifications. The library purification steps used the on-bead AMPure XP approach consisting of: AMPure XP Beads were added to the reaction and incubated for 5 minutes at room temperature, after incubation it was placed on a magnetic stand and after 5 minutes the supernatant was carefully removed and discarded. Next, 200 μl of 80% freshly prepared ethanol was added to the tube while in the magnetic stand. The reaction was incubated at room temperature for 30 seconds, and then the supernatant was carefully removed and discarded. Subsequently, 200 μl of 80% freshly prepared ethanol was added to the tube while in the magnetic stand. Next, the tube was incubated at room temperature for 30 seconds, and then the supernatant was carefully removed and discarded. The beads were then air dried for up to 5 minutes. The tube/plate was then removed from the magnet and DNA target was eluted from the beads by adding 22 μl of 10 mM Tris-HCl, pH 8.0 or 0.1×TE. Next, the tube was placed on a magnetic stand. After the solution is clear (about 5 minutes), 20 μl was transferred to a new PCR tube. For NEBNext End Repair, A-tailing, and adapter ligation enzyme and buffer volumes were adjusted to accommodate the on-bead AMPure XP purification strategy. Next, cfDNA libraries were amplified with Phusion Hot Start Polymerase.

The libraries were sequenced on an Illumina HiSeq 2000/2500 instrument using 100-bp paired-end reads at a depth of coverage of 1-2×. 5 Mb windows were used for evaluating cfDNA fragmentation patterns as this provided >20,000 reads per window at 1-2×genome coverage.

The fragments were mapped to their genomic origin and the fragment lengths evaluated in 504 windows of 5 Mb, covering ˜2.6 Gb of the genome. For each window, the fraction of small cfDNA fragments (100 to 150 bp) to larger cfDNA fragments (151 to 220 bp) were evaluated along with the overall coverage to obtain genome-wide fragmentation profiles for each sample. Machine learning was used to evaluate the variability of fragment lengths between normal and AD patient samples to identify profiles that differ from each other. The analysis identified that there was more variability (higher standard deviation) in the relative fragment lengths in AD patients compared to normal samples.

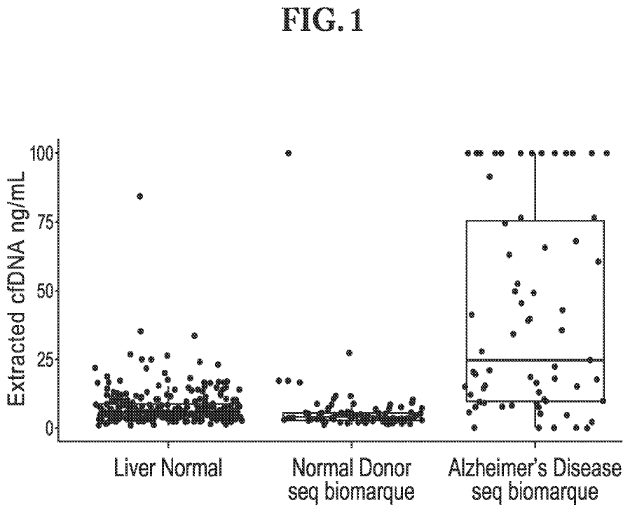

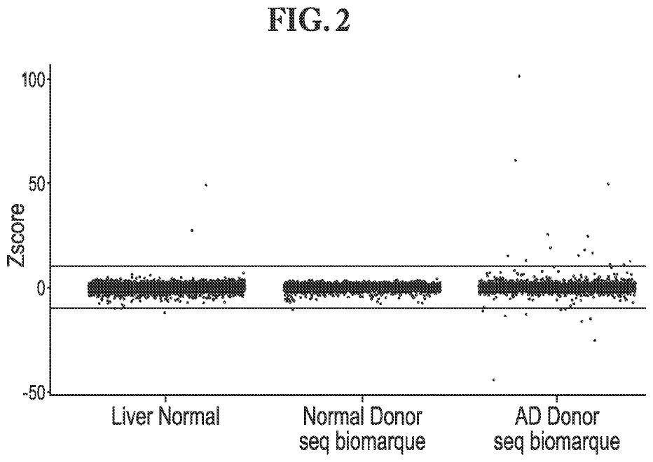

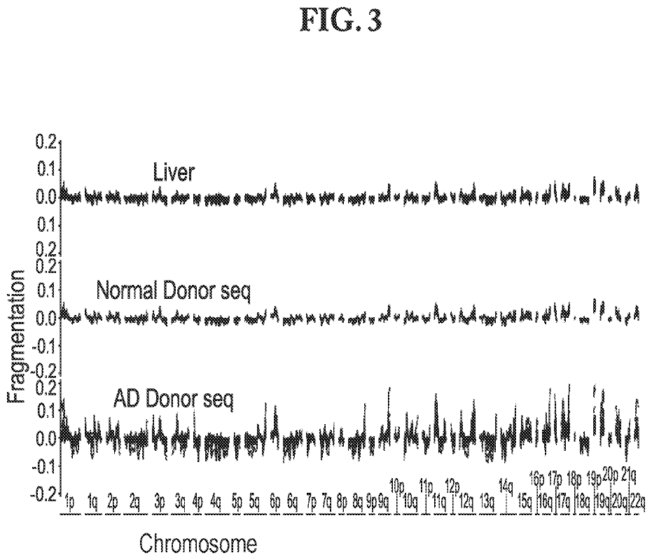

In addition to a higher variability in fragment lengths in AD patients compared to normal samples there was an increase in overall cfDNA concentration per mL in AD patients (FIG. 1). cfDNA from AD patients show a higher degree of variability in fragment size than what is seen in normal patients. Indeed, FIG. 3 indicates that there is a unique fragmentation pattern in the plasma of AD samples (lower panel) compared to the normal plasma samples (middle panel). Note that the cfDNA fragment profiles are consistent in “normal” plasma samples (peaks remain closer to 0). However, there are differences in the fragmentation profiles detected in the AD samples. In FIG. 2, each chromosome arm is labeled on the horizontal axis. The vertical axis graphs the ratio of short (˜100-150 bp) to long sequences (˜151-220 bp) of DNA in a given sample (the sample size is noted in the graph). Within each chromosome graph, the data illustrates the ratios in specific regions (windows) of the specific chromosome. The difference in fragment size variability can be utilized to identify patients with AD versus normal patients.

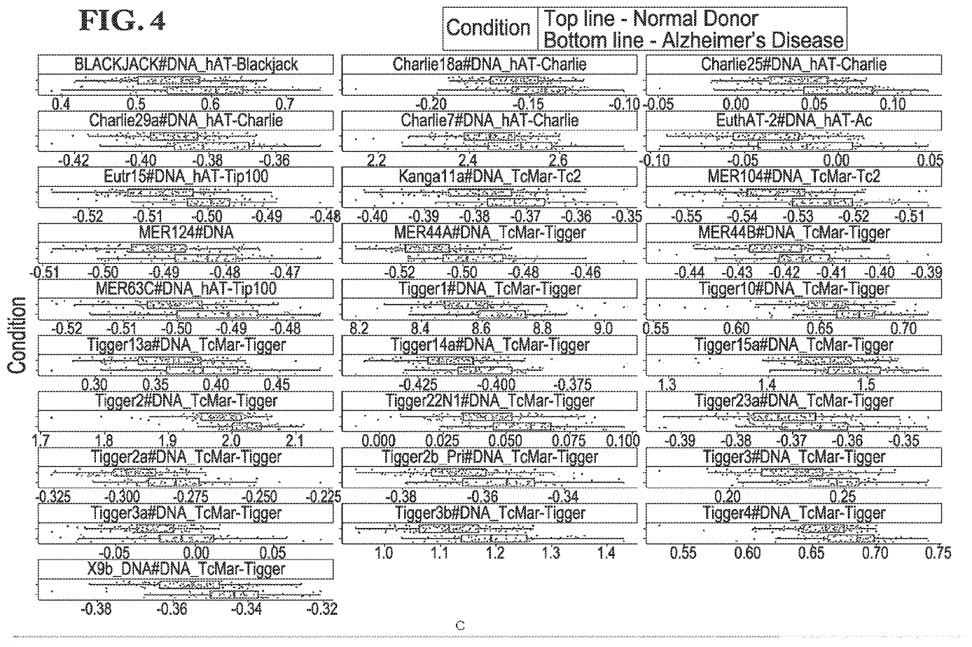

Additionally, aneuploidy was detected in the cfDNA of plasma AD specimens. FIG. 1 indicates that there may be increased detection of aneuploidy in the cfDNA from the plasma of AD samples. Furthermore, changes (i.e., increases) in the representation of DNA transposable elements was observed in the plasma of AD specimens (FIG. 4). Indeed, there was a significantly greater representation of 28 specific transposable elements that are significantly elevated (e.g, present in a higher amount) in AD plasma (versus control plasma).

These markers can be used in concert with other markers such as amyloid beta and tau to screen patients for AD to potentially increase the sensitivity of and specificity of AD detection.

### Example 2: Treatment of Alzheimer's Disease

A sample of 0.5 milliliters of cerebral spinal fluid (CSF) was obtained from a patient suspected of having Alzheimer's Disease. Whole genome sequencing (WGS) was then performed to analyze the DNA fragments in cell-free DNA (cfDNA) present in the CSF. Subsequently, the ratio of short (100 to 150 base pairs) to long (151 to 220 base pairs) fragments was quantified. The ratio of short to long fragments was determined to be 1.24 which was higher than the ratio obtained from a reference sample (e.g., a control sample from a subject without Alzheimer's Disease). Consequently, the patient was determined to have Alzheimer's Disease and was treated with an Alzheimer's treatment.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

In cases where numerical values are indicated in the context of the present disclosure, the skilled person will understand that the technical effect of the feature in question is ensured within an interval of accuracy, which typically encompasses a deviation of the numerical value given of ±10%, and preferably of ±5%. At the very least, and not as an attempt to limit the application of the doctrine of equivalents to the scope of the claims, each numerical parameter should at least be construed in light of the number of reported significant digits and by applying ordinary rounding techniques.

Unless otherwise indicated, all numbers expressing quantities of ingredients, properties such as molecular weight and median size, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified, thus fulfilling the written description of all Markush groups used in the appended claims.

Certain embodiments of this disclosure are described herein, including the best mode known to the inventor for carrying out the disclosure. Of course, variations on these described embodiments will become apparent to those of ordinary skill in the art upon reading the foregoing description. The inventor expects skilled artisans to employ such variations as appropriate, and the inventor intends for the disclosure to be practiced other than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entirety.

1. A method of treating a subject with an Alzheimer's Disease therapy, the method comprising:
a. obtaining a biological sample comprising cfDNA from the subject;
b. determining the length and amount of cell-free DNA (cfDNA) fragments in the biological sample; and
c. treating the subject with the Alzheimer's Disease therapy if the amount of short cfDNA fragments is less than or greater than the amount of long cfDNA fragments.

2. A method of treating a subject with an Alzheimer's Disease therapy, the method comprising:
a. obtaining a biological sample comprising cfDNA from the subject;
b. determining a ratio of short cfDNA fragments to long cfDNA fragments in the biological sample or determining the presence of aneuploidy in the cfDNA or determining the amount of transposable elements in the cfDNA; and
c. treating the subject with the Alzheimer's Disease therapy if the ratio of short cfDNA fragments to long cfDNA fragments in the biological sample is variable from a ratio of short cfDNA fragments to long cfDNA fragments in a reference sample, or
treating the subject with the Alzheimer's Disease therapy if the amount of aneuploidy in the cfDNA is variable from the amount of aneuploidy in a reference sample, or
treating the subject with the Alzheimer's Disease therapy if the amount of transposable elements in the cfDNA is variable from the amount of transposable elements in a reference sample.

3-4. (canceled)

5. The method of claim 1, wherein the subject has pre-clinical Alzheimer's Disease (AD), mild cognitive impairment (MCI), or AD.

6. The method of claim 1, wherein the reference sample is from a subject or subjects that does not have pre-clinical Alzheimer's Disease (AD), mild cognitive impairment (MCI), or AD.

7. The method of claim 1, wherein the biological sample is blood or plasma.

8. The method of claim 1, wherein the biological sample is cerebral spinal fluid.

9. The method of claim 1, wherein the short cfDNA fragments are about 100 to about 150 base pairs in length.

10. The method of claim 1, wherein the long cfDNA fragments are about 151 to about 220 base pairs in length.

11. The method of claim 4, wherein the transposable elements are selected from the group consisting of: BLACKJACK #DNA_hAT-Blackjack, Charlie18a #DNA_hAT-Charlie, Charlie25 #DNA_hAT-Charlie, Charlie29a #DNA_hAT-Charlie, Charlie7 #DNA_hAT-Charlie, EuthAT-2 #DNA_hAT-Ac, Eutr15 #DNA_hAT-Tip100, Kangalla #DNA_TcMar-Tc2, MER104 #DNA_TcMar-Tc2, MER124 #DNA, MER44A #DNA_TcMar-Tigger, MER44B #DNA_TcMar-Tigger, MER63C #DNA_hAT-Tip100, Tigger1 #DNA_TcMar-Tigger, Tigger10 #DNA_TcMar-Tigger, Tigger #13a #DNA_TcMar-Tigger, Tigger14a #DNA_TcMar-Tigger, Tigger15a #DNA_TcMar-Tigger, Tigger2 #DNA_TcMar-Tigger, Tigger22N1 #DNA_TcMar-Tigger, Tigger23a #DNA_TcMar-Tigger, Tigger2a #DNA_TcMar-Tigger, TIgget2b_Pri #DNA_TcMar-Tigger, Tigger3 #DNA_TcMar-Tigger, Tigger3a #DNA_TcMar-Tigger, Tigger3b #DNA_TcMar-Tigger, Tigger4 #DNA_TcMar-Tigger, and X9b_DNA #DNA_TcMar-Tigger.

12. A method for monitoring the progression of pre-clinical Alzheimer's Disease (AD), mild cognitive impairment (MCI), or AD in a subject in need thereof, the method comprising:
a. obtaining a first biological sample from the subject at a first time;
quantitating the length of cell-free DNA (cfDNA) fragments in the biological sample and/or a ratio of short cfDNA fragments to long cfDNA fragments in the first biological sample at the first time;
b. obtaining a second biological sample from the subject at a second time;
c. quantitating the length of cell-free DNA (cfDNA) fragments in the biological sample and/or a ratio of short cfDNA fragments to long cfDNA fragments in the second biological sample at the second time; and
d. determining that the pre-clinical Alzheimer's Disease (AD), mild cognitive impairment (MCI), or AD has progressed where the length of cell-free DNA (cfDNA) fragments in the second biological sample is variable from the length of cell-free DNA (cfDNA) fragments in the first biological sample or the ratio of short cfDNA fragments to long cfDNA fragments in the second biological sample is variable from the ratio of short cfDNA fragments to long cfDNA fragments in the first biological sample,
wherein the first time precedes the second time.

13-14. (canceled)

15. A method of diagnosing a neurodegenerative disease or disorder in a subject, the method comprising:
a. obtaining a biological sample from the subject; and
b. determining the length of cell-free DNA (cfDNA) fragments in the biological sample,
wherein the subject is diagnosed as having the neurodegenerative disease or disorder if the amount of short cfDNA fragments is variable from the amount of long cfDNA fragments.

16. (canceled)

17. The method of claim 15, wherein the neurodegenerative disease or disorder is pre-clinical Alzheimer's Disease (AD), mild cognitive impairment (MCI), or AD.

18. The method of claim 15, wherein the biological sample is blood.

19. The method of claim 15, wherein the biological sample is cerebral spinal fluid.

20. The method of claim 15, wherein the reference sample is from a subject that does not have pre-clinical Alzheimer's Disease (AD), mild cognitive impairment (MCI), or AD.
